(19) United States
(12) Patent Application Publication
(10) Pub. No.: US 2010/0226968 A1
(43) Pub. Date: Sep. 9, 2010

(54) Topical agent for muscle treatment

(76) Inventor: Eric Holgate, North Augusta, SC (US)

Correspondence Address: Kenneth Buffington, # 100, 4434 Columbia Rd., Martinez, GA, 30907, US

(21) Appl. No.: 12/381,057

(22) Filed: Mar. 7, 2009

(51) Int. Cl. A61K 9/127; A61K 33/06; A61P 21/02

(52) U.S. Cl. 424/450; 424/682

(57) ABSTRACT

A topical liposomal base delivery agent for physiologically enhancing muscle efficiency and relaxation, which combines the physiological effects of guaifenesin, magnesium, and methylsulfonylmethane. The resulting physiological reaction reduces muscle and joint soreness in the host. The present invention is an improvement over existing methods that deliver magnesium to muscle tissue.

### CROSS-REFERENCE TO RELATED APPLICATIONS

Not Applicable.

### FEDERALLY SPONSORED RESEARCH

Not Applicable.

### SEQUENCE LISTING OR PROGRAM

Not Applicable.

### BACKGROUND

1. Field of Invention

The present invention relates to topical methods and compositions for treating muscles by enhancing muscle efficiency and relaxation. The topical methods and compositions of the invention are especially suitable for relieving pain or discomfort from strained or damaged muscle tissue caused by physical activities. The invention is also useful in treating neuromuscular problems and exercise induced muscle cramps.

2. Prior Art

Magnesium ions are essential in all cells of all living organisms. Many enzymes require the presence of magnesium ions as a catalyst, especially enzymes utilizing phosphate transfer from ADP and ATP muscle contractility, and neuronal transmission of those enzymes which use other nucleotides to synthesize DNA and RNA. Magnesium inhibits nerve impulses and relaxes muscle contractions, thereby functioning antagonistically to calcium. Traditionally, magnesium enters the body as magnesium sulfate which is commonly available in dry form as epsom salts. Excess magnesium in the blood is freely filtered by the kidneys.

Guaifenesin is an expectorant drug sold over the counter and usually taken by mouth to assist expectoration in the treatment of coughing. A lesser know fact is that guaifenesin also acts as a skeletal muscle relaxant. Although it has not been proven, some believe that this skeletal muscle relaxation is achieved by depressing transmission of nerve impulses in the central nervous system.

Methylsulfonylmethane (MSM) is a form of sulfur that is found naturally in the human body. It has anti-inflammatory properties and supports healthy connective tissues such as tendons, ligaments, and muscle. Methylsulfonylmethane is effective for decreasing muscle soreness. Dimethyl sulfoxide (DMSO) has similar properties.

Liposomes are lipid vesicles made of membrane-like lipid bilayers separated by aqueous layers. Liposomes have been widely used to encapsulate biologically active agents for use as drug carriers since water or lipid soluble substances may be entrapped within the aqueous layers or within the bilayers themselves. There are numerous variables that can be adjusted to optimize this drug delivery system. These include, the number of lipid layers, size, surface charge, lipid composition and the methods of preparation.

U.S. Pat. No. 5,898,037 to Marx teaches novel pharmaceutical compositions which comprise magnesium compounds in hypertonic amounts. These compositions are formulated for local application for the treatment of conditions such as acne, arthritis, periodontal disease ophthalmic conditions (e.g., conjunctivitis), hemorrhoids, vaginal infections and inflammation, and ulcerative colitis.

U.S. Pat. No. 5,922,765 to Fleming teaches methods and compositions for the prevention and/or treatment of exercise-induced muscle cramps, stiffness, pain or spasms, using magnesium gluconate alone or in combination with one or more antioxidants or conventional pharmacologic therapy.

U.S. Pat. No. 6,537,590 to Lee teaches methods of making solutions for the treatment of osteoarthritis which include magnesium oxide; ethylenediaminetetraacetic acid; glycerin; and water.

U.S. Pat. Application No. 20050089581, filed Apr. 28, 2005, to Shealy teaches topical compositions and methods for restoring dehydroepiandrosterone (DHEA) levels in humans which include magnesium chloride, a suitable solvent, a gelling agent and a glycerin.

U.S. Pat. No. 6,887,492 to Kay, filed May 3, 2005, teaches an orally administered pharmaceutical composition that provides for the controlled release of magnesium. The composition has an interactive agent component and a magnesium component which has an enteric coating that controls the release of the magnesium until the composition is in the small intestine or colon.

U.S. Pat. No. 6,841,173 to Reynolds, filed Jan. 11, 2005, teaches chemical formulations and methods for reducing muscle and joint soreness. A representative chemical formulation includes compounds such as a selenium-compound, chondroitin sulfate, glucosamine, and/or methylsulfonylmethane. A representative method for reducing muscle and joint soreness includes administering the chemical formulation to a host.

U.S. Pat. Application No. 20050196434, files Sep. 28, 2005, to Brierre teaches a method of making a pharmaceutical composition and a method for the transdermal delivery of magnesium which includes a salt of zinc a vitamin such as B-complex vitamin, a carotenoid, a mineral, or a combination thereof may also be included in the transdermal pharmaceutical composition. A therapeutically effective amount of progesterone may also be included in the transdermal pharmaceutical composition.

U.S. Pat. No. 5,501,859 to Woods teaches a method of making an absorbable magnesium composition which includes a magnesium salt in a liposomal material encapsulating the magnesium salt.

None of these existing compositions are specifically formulated for topically enhancing muscle efficiency and relaxation. Furthermore, none of these compositions are specifically formulated for treating muscle cramps and soreness. These compositions are simply like other pills and capsules on the market that relieve muscle soreness, which when taken orally disperse throughout the blood stream providing relief to the damaged or affected area. These compositions were formulated as combinations of known anti-inflammatory agents, muscle relaxants and long used pain relievers. However, many of these compositions did not work because they were degraded in the digestive process and there were some unwanted side effects such as stomach upset associated with the products that did provide relief. Many of these existing compositions rely on oral magnesium which is slow acting and is primarily used as a laxative or aiding the body in the absorption of calcium. Furthermore, these existing compositions mostly only provide anesthetic relief or desensitize tissue without any physiological effect to the muscle. The object of the present invention provides a physiological reaction from magnesium in the muscle tissue. Therefore, there is a need for a topical base delivery agent developed to provide a physiological reaction for enhancing muscle efficiency and relaxation.

### SUMMARY OF THE INVENTION

According to its major aspects and broadly stated, the present invention is a liposomal base delivery agent for the treatment of muscle tissue with a combination of three main ingredients: guaifenesin, magnesium, and methylsulfonylmethane. The present invention is an improvement over methods that deliver magnesium to muscle tissue.

An important feature of the present invention is the method of delivery to the muscle tissue. Liposomal delivery is important as it temporarily disrupts the cell membrane to help with penetration. The present invention utilizes a topical based liposomal delivery system which allows the main ingredients to enter the muscle tissue directly and bypassing degradation by the liver. The liposome delivery system makes the topical base delivery agent fast acting, deep penetrating, and provides prolonged relief at the muscle. Also, the present invention physiologically reacts with skeletal muscle tissue, unlike other claimed muscle relaxants on the market. The base delivery agent may be in the form of a cream, gel, foam, or liquid; however, a cream is used in the present invention.

Other systems, methods, features, and advantages of the present invention will be or will become apparent to one with skill in the art upon examination of the following detailed description. It is intended that all such additional systems, methods, features, and advantages be included within this description, be within the scope of the present invention, and be protected by the accompanying claims.

### DETAILED DESCRIPTION

The present invention provides for chemical formulations and methods of use thereof directed towards reducing muscle and joint soreness. The chemical formulation includes compounds such as a magnesium compound, methylsulfonylmethane, and guaifenesin. Experiments show that the invention will achieve the intended results without guaifenesin, but optimally performs when guaifenesin is present in the formulation. The methods directed towards enhancing muscle efficiency include providing an effective amount of the chemical formulation to a host.

One embodiment of the chemical formulation includes combining a magnesium compound with methylsulfonylmethane. The formulation performs best with the addition of guaifenesin. Magnesium compounds, guaifenesin, and methylsulfonylmethane are effective for decreasing muscle soreness. Another embodiment of the chemical formulation includes combining magnesium compound, guaifenesin, and methylsulfonylmethane with a liposomal base delivery agent. The liposomal base delivery agent allows for direct application to the affected area resulting in lower dosages, faster action and less systemic effect. Guaifenesin mechanism of action is unknown, but it aids in thinning mucus secretions, transporting nutrients, and removing phosphates and possibly lactic acid from the muscle.

The magnesium compounds include, but are not limited to magnesium chloride hexahydate which provides the magnesium chloride as a major ingredient. Guaifenesin includes, for example, any pharmaceutically acceptable form but is most often obtained as a powder.

Methylsulfonylmethane (MSM) is a naturally occurring, organic, sulfur-containing compound that acts as a sulfur donor, and is also known as dimethyl sulfone. MSM also has anti-inflammatory effects within muscle fiber. The compounds used to produce the chemical formulation described herein may be administered as such, or in the form of a precursor compound from which the compound can be derived. In general, precursor compounds are derivatives of one of the compounds described herein, the pharmacological action of which results from the conversion by chemical or metabolic processes in vivo to the compound.

The chemical formulation of the present invention may be used as the active ingredient in combination with a pharmaceutically acceptable liposomal delivery system. As used herein, “liposomal delivery system” includes any and all topical base delivery agents which have liposomes to enhance drug delivery. The liposomal delivery system in the present invention may be in the physical form of any lipid cream, gel, liquid, foam, or aerosol. However, it is more efficient to simply use a commercially available off-the-shelf liposomal base delivery agent in the form of a cream. Except insofar as any liposomal delivery system is incompatible with the compounds used in practicing embodiments of the invention, such as by producing any undesirable biological effect or otherwise interacting in a deleterious manner with any other component(s) of the pharmaceutical composition, its use is contemplated to be within the scope of this invention. As indicated above, the chemical formulation is administered topically as a liposomal base delivery agent.

The chemical formulation described above may be administered using any amount and any route of administration effective for decreasing muscle and joint soreness. Thus, the expression “amount effective to reduce sore muscles and joints”, as used herein, refers to a nontoxic but sufficient amount of the chemical formulation to provide the desired reduction in muscle and joint soreness. The exact amount required will vary on the patient, depending on the age, weight, and general health of the individual patient, the severity of the soreness, the particular chemical formulation, and the like.

The chemical formulation typically includes about 5% to 15% of magnesium chloride by weight, about 2% to 10% of guaifenesin by weight, and about 2% to 10% of methylsufonylmethane by weight. These three ingredients are combined with a liposomal base delivery agent. The base delivery agent prescription grade formulation includes guaifenesin greater than 4%.

### Embodiment A

### EXAMPLE

TABLE 1
EXEMPLARY CHEMICAL FORMULATION
Quantity made:

| Ingredients | Quantity (approx. % by weight) |
|---|---|
| Magnesium Chloride USP | 10 |
| Dimethyl Sulfone Crystal | 5 |
| Guaifenesin USP Powder | 4 |
| Liposomal Base Q.S. | 100 |

Guaifenesin USP Powder must be 4% or less without a prescription.

Table 1 describes an embodiment of the chemical formulation as implemented into a liposomal cream base that can be topically administered to a host. The preferred embodiment of the cream formulation includes approximately 10 percent by weight of the magnesium compound.

The cream formulation can be topically administered into the host by externally rubbing a sufficient amount of the cream over the target muscle or muscle groups. Typically, about 1 to 2 grams of cream is a sufficient amount to reduce muscle and joint soreness; however, this varies with the size of the host and the target muscle group. A sufficient amount of cream is applied externally to cover the target muscle or muscle groups for treatment. Typically, a sufficient amount of cream is topically administered at least (3) three times per day or within a 24 hour period. Additional cream can be topically administered for hosts with severe muscle or joint soreness. However, the exact regimen for administration of the chemical formulation described herein may be dependent on the needs of the individual host and the exact concentration of the individual ingredients.

The above embodiments of the present invention are merely possible examples of implementations, set forth only for a clear understanding of the principles of the invention. Many variations and modifications may be made to the above embodiment(s) of the invention without departing substantially from the spirit and principles of the invention. It will be apparent to those skilled in the art that many changes and substitutions can be made to the preferred embodiment herein described without departing from the spirit and scope of the present invention as defined by the following claims.

What is claimed is:

1. A pharmaceutical composition for enhancing muscle efficiency and recovery, comprising:
(a) a therapeutically effective amount of a magnesium compound;
(b) a therapeutically effective amount of a sulfur donor compound;
(c) a therapeutically effective amount of skeletal muscle relaxant; and
(d) a pharmaceutically acceptable topical carrier.

2. The composition of claim 1, wherein said skeletal muscle relaxant is selected from a group consisting of guaifenesin, glyceryl quaiacolate, and methocarbonal.

3. The composition of claim 2, wherein said skeletal muscle relaxant is guaifenesin.

4. The composition of claim 1, wherein said sulfur donor compound is selected from a group consisting of organosulfur compounds.

5. The composition of claim 4, wherein said organosulfur compounds are selected from a group consisting of methylsulfonylmethane and dimethyl sulfoxide.

6. The composition of claim 5, wherein said sulfur donor compound is methylsulfonylmethane,

7. The composition of claim 1, wherein said acceptable topical carrier is a liposomal delivery system.

8. The composition of claim 7, wherein said liposomal delivery system is liposomal base delivery agent.

9 The composition of claim 8, wherein said liposomal base delivery agent is selected from a group wherein the physical form is a cream, gel, foam, liquid, and aerosol.

10. The composition of claim 9, wherein said liposomal base delivery agent is in the physical form of a cream.

11. The chemical formulation of claim 1 having: about 5% to 15% of magnesium chloride by weight chemical formulation, about 2% to 10% of guaifenesin by weight of chemical formulation, about 2% to 10% of methylsufonylmethane by weight chemical formulation; with all ingredients mixed in a liposomal base delivery agent.

12. A topical method of reducing muscle soreness and discomfort comprising administering an amount of the chemical formulation of claim 1 effective to reduce the muscle soreness and discomfort in a host.

13. The method of claim 12, wherein the amount effective to reduce muscle and joint soreness includes about 1 to 2 grams of topical base delivery agent administered about at least (3) three times per day or within a 24 hour period.

14. The method of claim 12, wherein the amount effective to reduce muscle and joint soreness includes a sufficient amount of topical base delivery agent to cover the target muscle or muscle group at least (3) three times per day/24 hour period.

15. The method of claim 12, wherein the amount effective to reduce muscle and joint soreness includes a sufficient amount of topical base delivery agent to externally cover the target muscle or muscle groups.
